(19) United States
(12) Patent Application Publication
(10) Pub. No.: US 2006/0106315 A1
(43) Pub. Date: May 18, 2006

(54) Guided hypodermic cannula

(76) Inventor: Roger Edens, Oconomowoc, WI (US)

Correspondence Address: DUANE MORRIS LLP;IP DEPARTMENT, 30 SOUTH 17TH STREET, PHILADELPHIA, PA, 19103-4196, US

(21) Appl. No.: 11/084,491

(22) Filed: Mar. 18, 2005

Related U.S. Application Data
(60) Provisional application No. 60/628,809, filed on Nov. 17, 2004.

(51) Int. Cl. A61B 8/00

(52) U.S. Cl. 600/461

(57) ABSTRACT

A ultrasound transducer and needle assembly has a needle having a lumen and a distal end having a point at a first radial location, and a support rod interior to the lumen. The ultrasound transducer and its support rod are fixed in the lumen at a second radial location, the second radial location being between about 135 and about 225 radially from the first radial location.

### CROSS-REFERENCE TO RELATED APPLICATIONS

This application claims the benefit of U.S. Provisional Patent Application No. 60/628,809, filed Nov. 17, 2004.

### FIELD OF THE INVENTION

The present invention is generally related to medical devices, and more particularly to devices used for the cannulation of blood vessels in which at least one ultrasonic transducer aids in the location of a target blood vessel.

### BACKGROUND OF THE INVENTION

The cannulation of blood vessels with a hypodermic needle is a common and well known medical procedure. While the insertion of a hypodermic needle into a blood vessel is often routine for a medical professional, vessels can be difficult to locate in many circumstances. For example, the location of a small blood vessel, such as those of small children, persons with unusual anatomy, and those of obese persons, is often quite difficult. Difficulty in locating a blood vessel and insertion of a hypodermic needle can result in unnecessary tissue damage, extreme discomfort to the patient, and often causes delays in the treatment of patients.

One of the devices developed to overcome this problem is the Doppler guided hypodermic needle. For example, in U.S. Pat. No. 4,887,606, issued to Yock, et al., incorporated herein by reference, a Doppler guided hypodermic needle is disclosed that includes an ultrasound transducer located within the lumen of the hypodermic needle. The transducer is capable of both emitting and receiving ultrasound signals. The hypodermic needle has a circular cross-sectional shape, with a centrally disposed longitudinal axis, and a beveled distal end that terminates in a sharp, off-axis point. The ultrasound signal that is emitted by the transducer is understood in the prior art to be centered on the longitudinal axis of the needle, with the signal strength decreasing rapidly with increased radial distance from the longitudinal axis of the needle. Significantly, the Doppler probe is free to move within the lumen of the needle relative to the longitudinal axis.

The ultrasound signals that are reflected by the patient's blood vessels, which may be a reflection from components of the blood flowing within the vessel or from motion of the vascular wall, provides a reflected signal that is received by the transducer. The signal is transmitted from the transducer through the probe's body located within the lumen of the hollow needle, to a device for interpretation and display of the signal. The reflected ultrasound signal can be presented to the medical professional in a variety of forms, including as an audio signal and a video representation, effectively providing feedback for the medical professional. The reflected ultrasound signal is commonly provided in audio form. The strength of the signal reflected from the blood vessel changes in response to the movement of the needle, depending upon whether the needle is approaching or receding from the blood vessel, and according to the well known Doppler effect. The medical professional will typically advance the needle toward the blood vessel, resulting in an increase in signal strength, until the needle is inserted. Relying upon the ultrasound signal as feedback related to the position of the needle relative to the target blood vessel, the medical professional can more accurately and quickly locate and cannulate the blood vessel.

A disadvantage associated with known Doppler probe devices of the foregoing type, is that the perceived change in signal strength does not always appear to correlate accurately with the position of the target blood vessel. Rather, although the medical professional may advance the needle continuously, in the same direction, with the signal strength gradually increasing to a maximum, the signal may pass through its maximum value, then begin to decrease in strength without the needle being inserted into a blood vessel. The understanding in the prior art has been that, if the needle is being advanced through tissue toward the blood vessel, the signal strength should increase to a maximum upon insertion of the needle point into the blood vessel. The discrepancy between this understanding and the phenomenon of signal strength peaking and decreasing without insertion, has created a significant problem in the art.

Referring to FIGS. 1, 1a and 1b, it is believed that a dependence exists between the direction of propagation of the ultrasound signal A and the position of the ultrasound transducer B within the central lumen C of a needle D having a point E. It has been found that an ultrasound signal A emitted from ultrasound transducer B propagates through a medium F generally with peak signal strength along an axis G centered on lumen C and emitter H. The strength of that ultrasound signal A decreases at greater radial distances I from axis G. When, as in the prior art, ultrasound transducer B is positioned at the same radial position as point E of needle D, or at a relatively small radial distance from point E, ultrasound signal A is emitted about another axis J that is not parallel to central longitudinal axis G of lumen C, and is indeed a significant radial distance away from longitudinal axis G. The ultrasound signal A appears to be reflected off portions of needle E, which results in a difference in angle between axis J of the propagating ultrasound signal A and central longitudinal axis G of needle D (FIG. 1b).

### SUMMARY OF THE INVENTION

An assembly of a needle and an ultrasound transducer is provided for use in the cannulation of blood vessels that includes a hollow needle having a point at a distal end, and an ultrasound transducer located at an internal portion of the needle so as to be radially spaced away from the point, often more than a radial distance. The needle often has a lumen with a substantially circular cross-section and a distal end having an axially extending point at a first radial location. The ultrasound transducer is positioned within the needle lumen where it may be fixed to the internal surface of the needle that defines the lumen at a second radial location, the second radial location being between about 135 and about 225 degrees from the first radial location. In this way, the ultrasound transducer is positioned at a location sufficiently distant from the location of the needle point that the direction of propagation of an ultrasound signal is substantially centered on the longitudinal axis of the needle.

In a further embodiment of the invention, a device for the cannulation of blood vessels is provided that includes a support rod having a flexible body and an ultrasound transducer located a distal end. Electrical conductors electrically are engaged between the ultrasound transducer and a source of power and signal receiving means at a proximal end of the support rod. A needle is provided that includes a lumen defined by an internal surface of the needle and which extends along a longitudinal axis to an opening at a distal end. The distal end of the needle has a point located at a first radial location relative to the longitudinal axis. Advantageously, the support rod is located adjacent to the internal surface at a second radial location.

In a further embodiment of the invention, a method of cannulation of a blood vessel, is provided that includes the steps of inserting an ultrasound transducer and needle assembly into tissue. The assembly includes a needle having a lumen defined by an internal surface of the needle that extends along a longitudinal axis to an opening at a distal end. The distal end has a point located at a first radial location relative to the longitudinal axis, with an ultrasound transducer located within the lumen at a second radial location relative to the longitudinal axis and spaced away from the first radial location. A signal is received from the ultrasound transducer, the signal having a characteristic identifying proximity to a blood vessel so that the needle may be moved toward and into a detected blood vessel based upon the signal characteristic.

### BRIEF DESCRIPTION OF THE DRAWINGS

These and other features and advantages of the present invention will be more fully disclosed in, or rendered obvious by, the following detailed description of the preferred embodiment of the invention, which is to be considered together with the accompanying drawings wherein like numbers refer to like parts and further wherein:

FIG. 1 is a side elevational view of a prior art needle positioned in a portion of a patients body;

FIG. 1a is an enlarged, longitudinal cross-sectional view of a distal end of the prior art needle and probe assembly shown in FIG. 1;

FIG. 1b is a further enlarged cross-sectional view of the distal end of the prior art needle and probe assembly shown in FIGS. 1 and 1a, illustrating the effect of the point of the needle upon ultrasound waves emanating from within the needle's lumen in accordance with the prior art;

FIG. 2 is a partially phantomed, perspective view of a needle and a guidance subassembly formed in accordance with one embodiment of the invention;

FIG. 3 is an enlarged, broken-away, cross-sectional view of the distal end of the needle and guidance subassembly shown in FIG. 2, illustrating the lack of effect of the point of the needle upon ultrasound waves emanating from within the needle's lumen when the ultrasound transducer is radially spaced from the point of the needle in accordance with one embodiment of the present invention;

FIG. 4 is an end view of the needle and guidance subassembly shown in FIG. 2;

FIG. 5 is a broken-away cross-sectional view of a distal portion of a ultrasound transducer support rod assembly;

FIG. 6 is an end view, similar to FIG. 4, of an alternate embodiment of needle and guidance subassembly;

FIG. 7 is an end view, similar to FIGS. 4 and 6, of a further alternate embodiment of needle and guidance subassembly;

FIG. 8 is a schematic view of the needle and guidance subassembly of FIG. 2 connected to a source of power and display means; and

FIG. 9 is a schematic view of a proximal end of needle and guidance subassembly illustrating one possible interconnection scheme for connection to a source of power and display means.

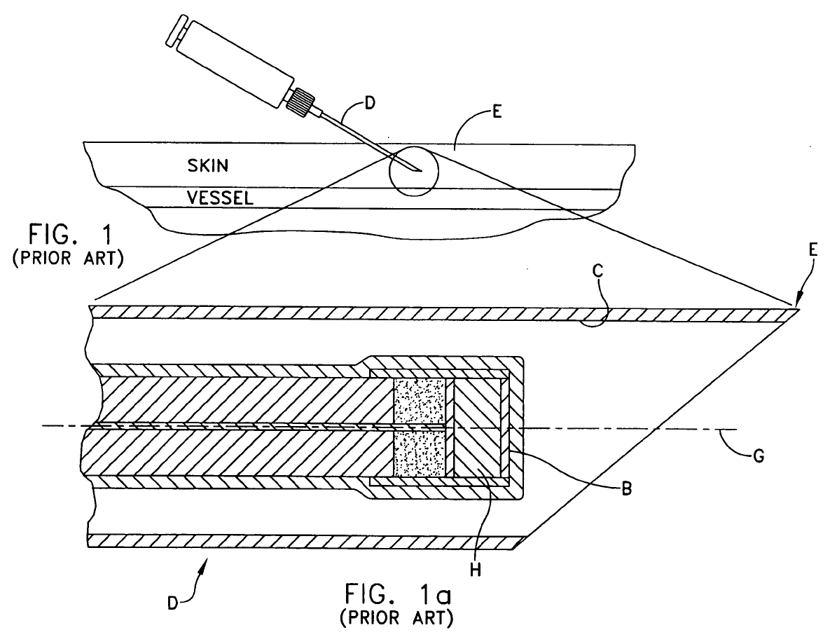

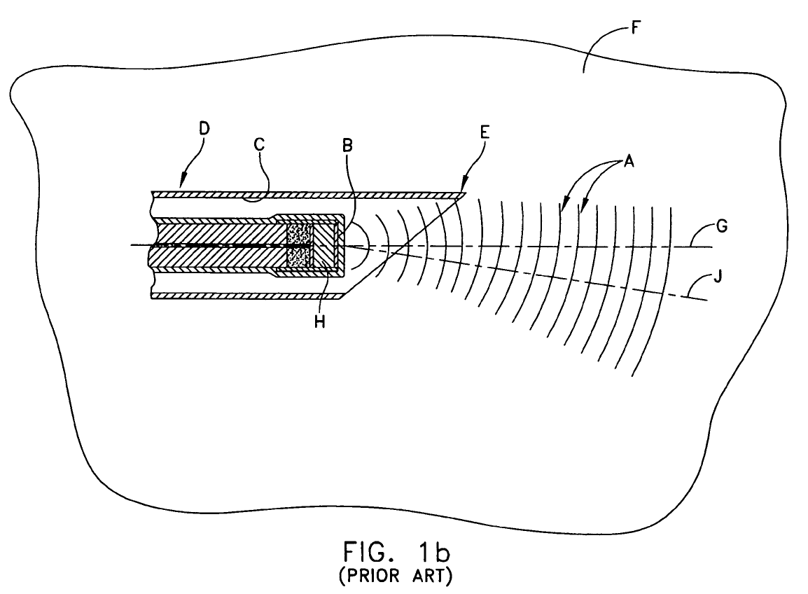

### DETAILED DESCRIPTION OF THE PREFERRED EMBODIMENT

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

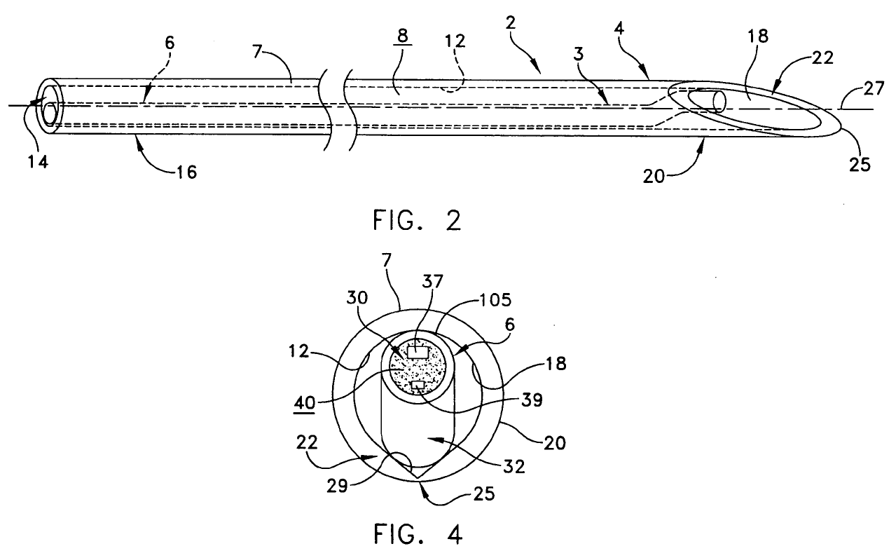

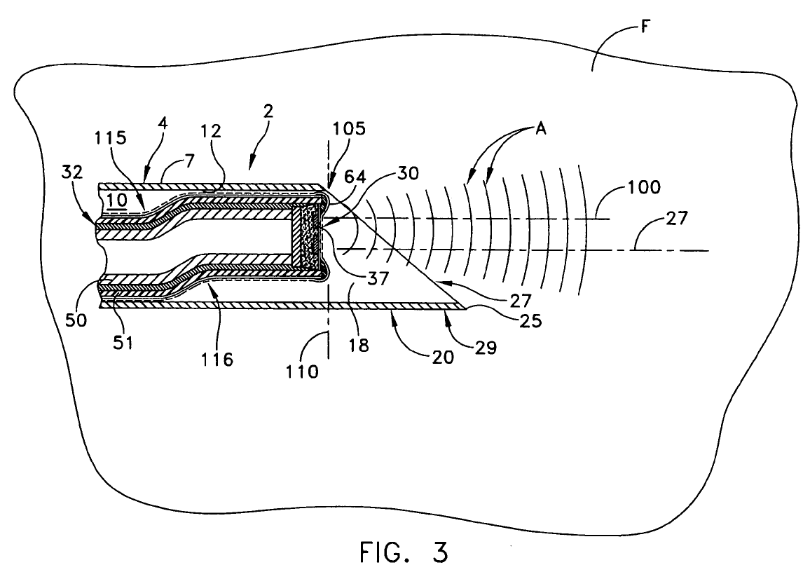

Referring to FIGS. 2 and 3, a guided hypodermic cannula 2 formed in accordance with one embodiment of the invention includes a needle 4 and a guidance subassembly 6. Needle 4 comprises a shaft 7 having an external surface 8 and an internal surface 10 which defines a longitudinally extending internal bore or lumen 12. Needle 4 may comprise any of the known hypodermic needles that are suitable for use in connection with cannulation of blood vessels. Stainless steel needles of various gauges may be employed, and may in principle be curving or straight. Lumen 12 includes an opening 14 at a proximal end 16 and an opening 18 at a distal end 20. A beveled and sharpened lance 22 includes a point 25 that projects outwardly from distal end 20 and is suitable for penetrating the skin of a subject. Point 25 is arranged in a radially “off-set” or “off-axis” relationship to a central longitudinal axis 27 of lumen 12 so as to define a first radial location 29 along shaft 7 (FIG. 4).

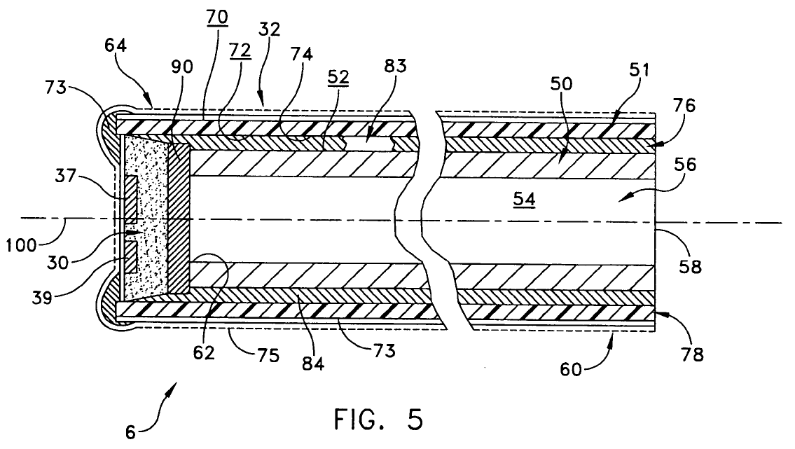

Referring to FIGS. 3, 5, 8, and 9, guidance subassembly 6 includes an ultrasound transducer 30, a support rod 32, and a signal routing and processing system 35, which are arranged and interconnected so as to provide a signal to ultrasound transducer 30, and to transmit a signal from ultrasound transducer 30 to suitable electronics of the type known in the art for processing, interpreting, and displaying information that is representative of ultrasound signals. More particularly, ultrasound transducer 30 may be selected from any one of a large number of commercially available ultrasound transducers available in the market. Such ultrasound transducers 30 will normally include both an ultrasound emitter 37 and an ultrasound detector 39 arranged together and located on a distal surface 40 of a single housing (FIGS. 4 and 5). Electrodes form a portion of the transducer and are conventionally arranged so as to receive power as well as transmit an electronic signal corresponding to a detected ultrasonic signal. Typically, ultrasound transducers 30 of the preferred type comprise a cylindrical disk, with a transmitting electrode on one surface and a power electrode on another surface. Such transducers often emit in a frequency range from about two MHz to about thirty MHz, although other frequency ranges may be selected for use with the present invention with adequate results.

Support rod 32 often comprises a pair of coaxially arranged elongate tubes 50, 51. More particularly, inner tube 50 is formed from stainless steel or the like, and includes an external surface 52 and an internal surface 54 which defines a longitudinally extending internal bore or lumen 56. Lumen 56 includes an opening 58 at a proximal end 60 and an opening 62 at a distal end 64. Outer tube 51 is often formed from a polymer, e.g., polyamide, and also includes an external surface 70 and an internal surface 72 which defines a longitudinally extending internal bore or lumen 74. Lumen 74 includes an opening 76 located at a proximal end 78 of outer tube 51. When assembled to one another a space 83 is defined between external surface 52 of inner tube 50 and internal surface 72 of outer tube 51. A clear epoxy, e.g., Epotech 84 or the like, often fills space 83 so as to fix the coaxial relation between tubes 50,51. External surface 70 of outer tube 51 further includes a highly conductive coating 73 along its entire length, e.g., gold or silver plating, which is then coated with a dielectric polymer sleeve 75, e.g., polystyrene.

When fully assembled, support rod 32 also includes an electrical conductor 90 located at distal end 64, which may be in the form of a conductive epoxy, e.g., silver epoxy or the like. Electrical conductor 90 is provided on the distal edge surface of inner tube 50, adjacent to opening 62 at distal end 64. Inner tube 50 and electrical conductor 90 are rigidly attached to an electrode on the proximal side of ultrasound transducer 30. Ultrasound transducer 30 has an outer diameter that is somewhat larger than the diameter of lumen 56. The distal end of outer tube 51 (adjacent to distal end 64 of inner tube 50) includes a portion of highly conductive coating 73 which is conductively bonded and thereby electrically interconnects to an electrode on a distal surface of ultrasound transducer 30 to highly conductive coating 73 so as to complete the circuit.

Referring to FIG. 3, support rod 32 is positioned within distal end 20 of needle 4 so as to substantially avoid interaction between ultrasound signals emitted from emitter 37 and the surrounding portions of needle 4, particularly point 25. The location of ultrasound transducer 30 within lumen 12 of needle 4 greatly affects the direction of propagation of an ultrasound signal through a given medium. A dependence has been identified between the direction of propagation of an ultrasound signal A and the position of ultrasound transducer 30 within lumen 12 relative to point 25. It has been found that ultrasound signal A propagates through medium F generally with peak signal strength along an axis 100 centered on emitter 37. The strength of that ultrasound signal decreases at greater radial distances from axis 100.

If, as in the prior art, ultrasound transducer 30 were to be located at the same radial position as point 25 of needle 4, or at a relatively small radial distance from point 25, an ultrasound signal would be emitted about an axis that is not parallel to central longitudinal axis 27 of lumen 12, and would indeed be at a significant angular relation to central longitudinal axis 27. In the present invention, ultrasound transducer 30 is disposed at a location sufficiently radially distant from point 25, i.e., off-set radially from central longitudinal axis 27 by as much as a diameter length, so that the direction of propagation of ultrasound signal A emitted from emitter 37 is substantially parallel with central longitudinal axis 27 of lumen 12 (FIG. 3).

In a preferred embodiment of the invention, a portion of distal end 64 of support rod 32 is fixed in position in contact with internal surface 10 which defines lumen 12 of needle 4. By fixed, it is meant that any range of motion of distal end 64 relative to distal end 20 of needle 4 is restricted such that distal end 64 of support rod 32 remains engaged with internal surface 10, and within a limited range of positions relative to central longitudinal axis 27. Preferably, distal end 64 is located within lumen 12 so as to be in an “off-set” or “off-axis” relationship to central longitudinal axis 27 of lumen 12 thereby defining a second radial position 105 along needle 4 that is between about 135° and 225° from first radial position 29 at point 25 (FIG. 4). Most advantageously, second radial position 105 is about 180 degrees from first radial location 29 relative to central longitudinal axis 27. In other words, ultrasound transducer 30 is positioned on the opposite side of internal surface 10 from point 25, which is sufficiently radially distant from point 25, i.e., off-set radially from central longitudinal axis 27, so that the direction of propagation of ultrasound signal A emitted from emitter 37 is substantially parallel with central longitudinal axis 27 of lumen 12 (FIG. 3). The longitudinal position of distal end 64 is preferably close to the intersection of opening 18 and beveled and sharpened lance 22, but not extending beyond the plane of intersection (shown generally at reference numeral 110 in FIG. 3).

The optimal radial and longitudinal location of distal end 64 within lumen 12 of a particular needle 4, and its limits, may be determined empirically by one of ordinary skill in the art by adjusting the position of support rod 32 along internal surface 10 to compensate for manufacturing induced tolerance variations between needles. In this way, deviations in the direction of propagation of ultrasonic energy relative to central longitudinal axis 27 of needle 4 may be observed. The extent to which deviations in ultrasonic signal strength become undesirable may be empirically determined by those of skill in the art, e.g., by immersion of support rod 32 in a suitable medium and applying suitable known imaging techniques, such as schlieren photography or the like.

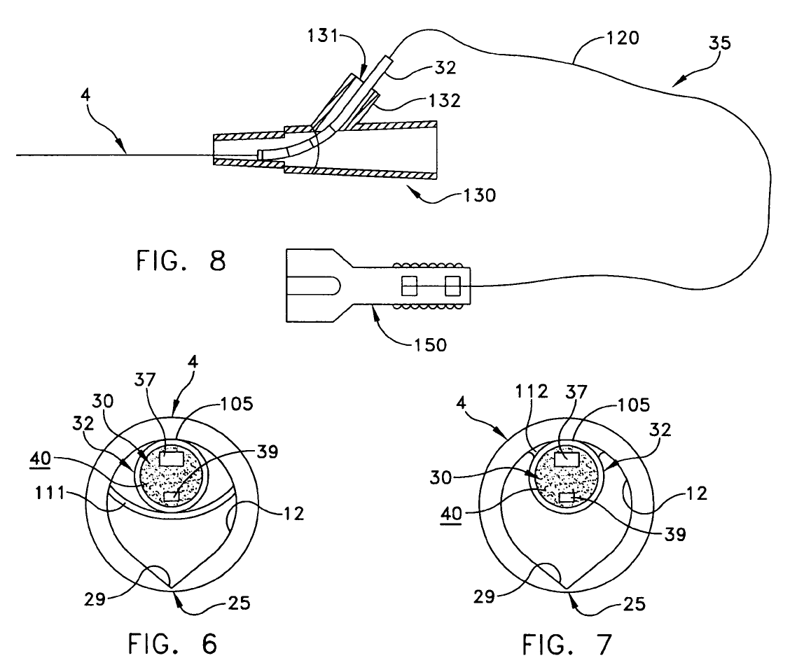

The preferred orientation of distal end 64 may be achieved by any suitable structure as long as it is adapted to maintain distal end 64 fixed at a location sufficiently radially distant from point 25 of needle 4 so as to provide ultrasound signal output at a prescribed relationship to central longitudinal axis 27. For example, a needle 4 and substantially straight support rod 32 may be employed, with distal end 64 fixed at second radial location 105 by a bracket 111 that extends across lumen 12 and between portions of internal surface 10 so as to support and maintain support rod 32 at second radial location 105 (FIG. 6). Alternatively, an adhesive 112 may be applied to the external surface of distal end 64 so as to support and maintain support rod 32 at second radial location 105 (FIG. 7). In another embodiment, the preferred orientation of distal end 64 may be achieved by introducing a first radiused bend 115 at a location along support rod 32 proximal to distal end 64 and a second radiused bend 116 at another location along support rod 32 proximal to distal end 64 (FIGS. 2, 3, and 4). Support rod 32 is often sufficiently elastic that first radiused bend 115 and a second radiused bend 116 together form a spring-like structure that helps to maintain distal end 64 of support rod 32 at the appropriate location within lumen 12, i.e., in or around second radial position 105, without the need for adhesives or additional mechanical structures.

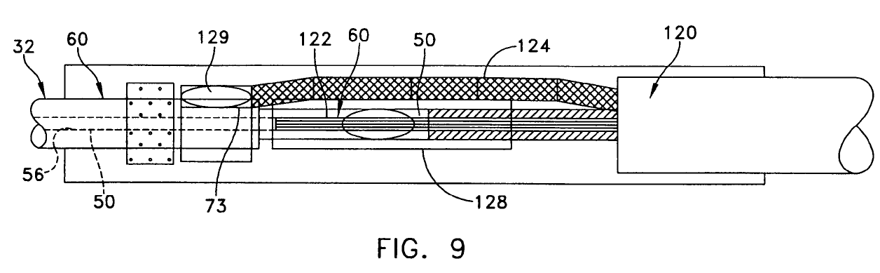

Referring to FIGS. 8 and 9, signal routing and processing system 35, includes a coaxial cable 120 having a centrally located signal conductor 122 and a braided ground or return 124. Signal conductor 122 is electrically engaged with proximal end 60 of inner tube 50 of support rod 32 by, e.g., solder or conductive adhesive. An insulating layer 128 often surrounds the portion of signal conductor 122 that is electrically engaged with proximal end 60 of inner tube 50. Insulating layer 128 may be a polymer, such as the polyether-block co-polyamiold by Atofina Chemicals, Inc., of Philadelphia, Pa., under the brand PEBAXde polymers S®, or other suitable insulating material. Braided ground 124 is electrically engaged with coating 73, for example by silver epoxy 129 or the like.

Signal routing and processing system 35 also includes an assembly 130 that receives needle 4 with support rod 32 electrically engaged with coaxial cable 120 and extending through distal port 131 and side arm 132. A proximal end of coaxial cable 120 is connected to a source of power 150 and is adapted to receive and communicate signals that are representative of the ultrasound signals received by ultrasound detector 39.

Needle 4 having a fully assembled support rod 32 positioned within lumen 12 according to the invention, i.e., so as to be located at second radial location 105, may be used as follows. Point 25 of needle 4 is inserted into subcutaneous tissue F of a patient (FIG. 3). Ultrasound transducer 30 is activated while needle 4 is moved in a circular motion, using point 25 as a pivot. Signals emitted and received by ultrasound transducer 30 and provided to signal routing and processing system 35 provide a medical professional with data that includes characteristics of signals reflected from a blood vessel. These reflected signals are often characterized as “strong”, i.e., corresponding to an indication that point 25 is moving substantially directly toward a target blood vessel or “weak” corresponding to an indication that point 25 is moving substantially away from a target blood vessel. Advantageously, due to the relative position of ultrasound transducer 30 at second radial position 105 within lumen 12, ultrasound signals Atravel along an axis that is substantially parallel to central longitudinal axis 27 of needle 4 thus ensuring that the indication that point 25 is moving substantially directly toward a target blood vessel is true. For example, signal routing and processing system 35 may convert the reflected ultrasound signals received from ultrasound transducer 30 to distinctive sounds, with a louder sound corresponding to a stronger signal and a softer sound corresponding to a weaker signal. Needle 4 is advanced in a direction corresponding to “strong” or an increasingly strong signal meaning that needle 4 is advancing toward and getting closer to the target blood vessel. If the signal weakens, at any time, the medical professional may stop advancing needle 4, and rotate it slightly to find the direction in which the signal is stronger.

It is to be understood that the present invention is by no means limited only to the particular constructions herein disclosed and shown in the drawings, but also comprises any modifications or equivalents within the scope of the claims.

What is claimed is:

1. Apparatus for use in the cannulation of blood vessels comprising:
a hollow needle having a point at a distal end; and
an ultrasound transducer located at an internal portion of said needle so as to be radially spaced away from said point by more than one radius.

2. An apparatus according to claim 1 wherein a support rod supports said ultrasound transducer, and a portion of said support rod contacts an internal portion of said needle so as to be radially spaced away from said point by more than one radius.

3. An apparatus according to claim 2 wherein said portion of said support rod that contacts said internal portion of said needle is between about 135 and about 225 from said point relative to a longitudinal axis of said needle.

4. An apparatus according to claim 2 wherein said portion of said support rod that contacts said internal portion of said needle is about 180 from said point relative to said longitudinal axis.

5. An apparatus according to claim 2 wherein said support rod is elongated and substantially rigid and comprises a distal end, a proximal end, and an intermediate portion, said distal end supporting said an ultrasound transducer and being in contact with an internal surface of said needle at between about 135 and about 225 from said point relative to a longitudinal axis of said needle, said proximal region being in contact with said internal surface.

6. An apparatus according to claim 2 comprising a bracket extending across a lumen defined within said needle, said bracket being located between portions of an internal surface that defines said lumen so as to support and maintain said support rod at between about 135 and about 225 from said point relative to a longitudinal axis of said needle.

7. An apparatus according to claim 4 wherein said support rod is fixed within said needle by an adhesive.

8. An ultrasound transducer and needle assembly comprising:
a needle including a lumen defined by an internal surface of said needle that extends along a longitudinal axis to an opening at a distal end, said distal end having a point located at a first radial location relative to said longitudinal axis;
a support rod positioned within said lumen and having an ultrasound transducer located on a distal end, said distal end of said support rod being substantially fixed to said internal surface at a second radial location.

9. An assembly according to claim 8 wherein said second radial location is between about 135 and about 225 from said first radial location and relative to said longitudinal axis.

10. An assembly according to claim 8 wherein said second radial location is about 180 from said first radial location and relative to said longitudinal axis.

11. An assembly according to claim 8 wherein said support rod is elongated and substantially rigid and comprises a distal end, a proximal end, and an intermediate portion, said distal end being in contact with said internal surface of said lumen at said second radial location, said proximal region being in contact with said lumen at a location radially opposite said second radial location, and said intermediate portion joining said distal and proximal ends.

12. An assembly according to claim 8 comprising a bracket extending across said lumen and between portions of said internal surface so as to support and maintain said support rod at said second radial location.

13. An assembly according to claim 8 wherein said support rod is fixed in said second radial location by an adhesive.

14. A device for the cannulation of blood vessels comprising:
a support rod having a flexible body and an ultrasound transducer located a distal end, electrical conductors electrically engaged between said ultrasound transducer and a source of power and signal receiving means at a proximal end of said support rod;
a needle including a lumen defined by an internal surface of said needle that extends along a longitudinal axis to an opening at a distal end, said distal end of said needle having a point located at a first radial location relative to said longitudinal axis wherein said support rod is located adjacent to said internal surface at a second radial location.

15. An assembly according to claim 14 wherein said second radial location is between about 135 and about 225 from said first radial location and relative to said longitudinal axis.

16. An assembly according to claim 14 wherein said second radial location is about 180 from said first radial location and relative to said longitudinal axis.

17. An assembly according to claim 14 wherein said support rod is elongated and substantially rigid and comprises a distal end, a proximal end, and an intermediate portion, said distal end being in contact with said internal surface of said lumen at said second radial location, said proximal region being in contact with said lumen at a location radially opposite said second radial location, and said intermediate portion joining said distal and proximal ends.

18. An assembly according to claim 14 comprising a bracket extending across said lumen and between portions of said internal surface so as to support and maintain said support rod at said second radial location.

19. An assembly according to claim 14 wherein said support rod is fixed at said second radial location by an adhesive.

20. Apparatus for use in cannulation of blood vessels comprising:
a hollow needle having a point located at a distal end;
an ultrasound transducer positioned within said needle for transmitting and receiving ultrasonic signals through said distal end;
a support rod for supporting said ultrasound transducer, wherein a portion of said support rod contacts an internal portion of said needle so as to be radially spaced away from said point; and
coaxial electrical conductors associated with said support rod for transmitting electrical signals to and from said ultrasound transducer, including a central conductor extending through said support rod electrically connected with a electrode located on said ultrasound transducer, and a conductor on a surface of said rod electrically interconnected with another electrode on said ultrasound transducer.

21. A method of cannulation of a blood vessel, comprising the steps of:
inserting into tissue an ultrasound transducer and needle assembly including a needle including a lumen defined by an internal surface of said needle that extends along a longitudinal axis to an opening at a distal end, said distal end having a point located at a first radial location relative to said longitudinal axis, and an ultrasonic transducer located within said lumen at a second radial location relative to said longitudinal axis;
receiving a signal from said ultrasound transducer, said signal having a characteristic identifying proximity to a blood vessel; and
moving said needle toward and into a detected blood vessel based upon on said signal characteristic.

22. A method according to claim 21 including locating said ultrasound transducer at a second radial location that is between about 135 and about 225 from said first radial location and relative to said longitudinal axis.
